(19) United States
(12) Patent Application Publication
(10) Pub. No.: US 2012/0309761 A1
(43) Pub. Date: Dec. 6, 2012

(54) FAST-RESPONSE PHOTOCHROMIC NANOSTRUCTURED CONTACT LENSES

(75) Inventors: Edwin Pei Yong Chow, Singapore (SG); Jackie Y. Ying, Singapore (SG); Yuri Shona Pek, Singapore (SG)

(73) Assignee: Agency for Science, Technology and Research, Connexis (SG)

(21) Appl. No.: 13/387,014

(22) PCT Filed: Oct. 27, 2010
(86) PCT No.: PCT/US2010/054244

§ 371 Date: Aug. 8, 2012

Related U.S. Application Data
(60) Provisional application No. 61/255,474, filed on Oct. 27, 2009.

(51) Int. Cl. G02B 5/23; A61K 31/5377; A61P 27/02; A61K 47/32

(52) U.S. Cl. 514/236.2; 252/586; 514/772.4

(57) ABSTRACT

A bicontinuous microemulsion of water, a monomer, and a surfactant copolymerizable with the monomer is polymerized to form a polymeric material, the polymeric material comprising a polymer matrix defining interconnected pores. The polymeric material may additional comprise at least one photochromic agent. The photochromic agent may be dispersed in one or both of the polymer matrix or the interconnected pores. The polymeric material may be used to form photochromic articles including ophthalmic articles such as contact lenses.

### FIELD OF THE INVENTION

The present invention relates generally to methods, compositions, and articles comprising photochromic polymeric materials, and particularly for use of these materials in contact lenses.

### BACKGROUND OF THE INVENTION

Photochromic compounds undergo a color change upon irradiation, and the photoproduct can be reversed back to the initial state thermally and/or by subsequent irradiation at a suitable wavelength of light. This interesting effect can be used in applications such as ophthalmic lenses, nonlinear device components, optical waveguides and shutters, light modulators, optical storage media and delay generators, as well as other optical devices depending on the response time and other properties of the photochromic compounds.

The use of photochromic compounds to produce a variety of tinted articles is known. For example, photochromic spectacles have found some success, providing the wearer the convenience of visible light absorbing lenses (sunglasses) only when exposed to bright light (e.g., daylight). Under low light conditions, the lenses are generally substantially colorless and provide optimal night and indoor vision. Photochromic spectacles eliminate the need for switching between sunglasses and regular spectacles.

However, existing photochromic compounds and methods for producing very thin photochromic articles, such as contact lenses, have been met with limited success. In some cases, the articles do not provide enough darkening to produce a noticeable difference to the wearer, and/or the existing photochromic compounds and manufacturing methods are not compatible with the materials and/or processes used for ophthalmic devices. In addition, the timescale of the thermal back-fading of the colored form of the photochromic compound to the colorless form is usually minutes to hours, which is too slow for certain applications.

Accordingly, improved compositions, methods, and articles are needed.

### SUMMARY OF THE INVENTION

The present invention relates generally to photochromic polymeric material, and related methods and articles. The subject matter of the present invention involves, in some embodiments, interrelated products, alternative solutions to a particular problem, and/or a plurality of different uses of one or more compositions and/or methods.

In some embodiments, the present invention provides a method of forming a polymeric material, comprising polymerizing a bicontinuous microemulsion comprising water, a monomer, and a surfactant copolymerizable with said monomer, to form a porous polymeric material comprising a polymer matrix defining interconnected pores at least partially filled by water, wherein said microemulsion further comprises a photochromic agent.

In some embodiments, the present provides a photochromic polymeric material for use in an ophthalmic device, comprising a polymer matrix defining interconnected pores, said interconnected pores containing water and said polymer matrix being substantially hydrophobic, and wherein the polymeric material further comprises a photochromic agent.

In some embodiments, the present invention provides an optical device comprising a photochromic agent rendering the device switchable from a first, relatively transparent state to a second, at least partially opaque state, whereby transmission of visible light through the optical pathway can change by at least 50 percent upon switching of the device from the first state to the second state, and from the second state to the first state, each within a period of time of no more than 30 seconds upon exposure to appropriate electromagnetic radiation and/or thermal relaxation.

### BRIEF DESCRIPTION OF THE DRAWINGS

Other aspects, embodiments, and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions, will control.

FIG. 1 shows a schematic of a contact lens, according to a non-limiting embodiment.

FIG. 2 illustrates a non-limiting structure of a bicontinuous microemulsion.

FIGS. 3-6 show schematic diagrams illustrating a non-limiting method for forming a contact lens from a bicontinuous microemulsion.

FIG. 7 shows non-limiting examples of pinhole lenses.

FIG. 8 shows the structure of a non-limiting photochromic agent, 6′-(2,3-dihydro-1H-indole-1-yl)-1,3-dihydro-3,3-dimethyl-1-propyl-spiro[2H-indole-2,3′-(3H)-naphtho(2,1-b)(1,4)oxazine (SPO), and one of the corresponding open forms.

FIGS. 9A-9C show field emission scanning electron microscopy graphs of non-limiting example of polymeric materials of the present invention.

FIG. 10 shows changes in the absorbance spectra of a polymeric material of the present invention comprising SPO upon UV irradiation for various time periods, according to a non-limiting embodiment.

FIG. 11 shows graphs of the absorbance as a function of time for the coloration and decoloration of a polymeric material of the present invention comprising SPO, according to a non-limiting embodiment.

FIG. 12 shows time-dependent photocoloration and bleaching of a polymeric material of the present invention comprising SPO, according to a non-limiting embodiment.

FIG. 13 shows a graph of tensile strength and tensile modulus of materials according to some embodiments.

### DETAILED DESCRIPTION

The present invention relates generally to photochromic polymeric materials, and related methods and articles. In some embodiments, an article is an ophthalmic lens, more particularly, a contact lens. A polymeric material may comprise a polymer matrix defining a plurality of interconnected pores. A photochromic agent may be substantially contained in the polymer matrix. In some cases, an ophthalmic lens may be used to protect eyes from strong light (e.g., UV-light).

Photochromic materials are materials which change color upon exposure to light (e.g., UV-light). Upon exposure to light, a photochromic material changes from a first colored state (e.g., colorless) to a second colored state (e.g., darkened), which is referred to as direct chromism. The reverse transition is referred to as reverse photochromism. In some cases, reverse chromism may be may be accelerated by heating. In addition, reverse photochromism may be inhibited due to stabilizing interactions of a photochromic agent with a polymer matrix or other component or material. Advantageously, the photochromic polymeric materials described herein may exhibit rapid reverse photochromism as compared to currently known materials. Without wishing to be bound by theory, this may be due, at least in part, to the ability to control the nano- or micro-environment surrounding the photochromic agent. The microenvironment may be controlled by varying the components provided in a bicontinuous microemulsion, as described herein. In some cases, the nano- or micro-environment may have an effect on the fast and ultrafast events undergone by a trapped photochromic agent. Generally, the degree of confinement of a photochromic agent may be affected by various factors including the structure, the orientation of the photochromic agent, the rigidity of the complex, and the polarity of a nano- or microcavity. The relatively hydrophilic interior and hydrophobic exterior of the molecular pockets may aid in providing a suitable and facilitating host for a photochromic agent, and additionally may offer a unique opportunity for studying size-controlled nano- and microenvironment effects such as reduced degrees of freedom of the photochromic agents.

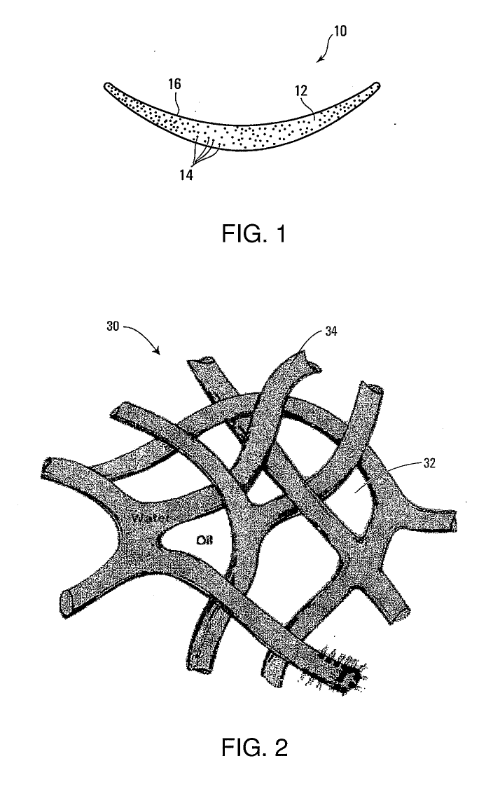

In some embodiments, a photochromic polymeric article of the present invention comprises a polymeric material and a photochromic agent, wherein the polymeric material comprises a polymer matrix defining a plurality of interconnecting pores. The term, “polymeric material,” as used herein, refers to a material comprising a polymer matrix and a plurality of interconnecting pores. For example, a non-limiting example of a contact lens of the present invention is depicted in FIG. 1. Contact lens 10 is formed of a porous polymeric material 12 and comprises a photochromic agent 14. The pores may be interconnected when at least some of them are joined or linked with each other to form one or more continuous networks. The pores may be filled with a fluid such as water, air, or another fluid. The fluid may be releasable from the polymeric material.

In some embodiments, a polymeric material may be formed from a bicontinuous microemulsion. A bicontinuous microemulsion may comprise water, a monomer, and a surfactant copolymerizable with the monomer, and optionally, a photochromic agent. The bicontinuous microemulsion may be polymerized to form a polymer matrix defining interconnected pores. For example, a polymer matrix may be prepared by polymerizing a bicontinuous microemulsion of one or more copolymerizable monomers, one or more surfactants copolymerizable with at least one of the monomers, and water, such that the resulting polymeric material has interconnected pores filled with water. The bicontinuous microemulsion may also include a polymerization initiator or a cross-linker, or both.

An exemplary structure of a bicontinuous microemulsion 30 is illustrated in FIG. 2, wherein oil domains 32 (containing the monomers) and aqueous domains 34 (containing water) are randomly distributed and respectively interconnected, extending in all three dimensions. When oil domains 32 are polymerized, the presence of the aqueous domains 34 results in interconnected pores filled with the water that was present in the aqueous domains 34.

In some cases, a method of farming a polymeric material comprises polymerization a bicontinuous microemulsion comprising a first continuous phase comprising water and a second continuous phase comprising a monomer and a surfactant copolymerization with said monomer to form a porous polymeric material. The polymeric material may comprise a polymer matrix portion formed from the second phase and a water portion (e.g., aqueous domains) formed from the first phase, the water phase forming interconnected pores defined in the polymer matrix. The polymeric material may comprise at least one photochromic agent. The photochromic agent may be dispersed in the first and/or second continuous phase prior to polymerization. Alternatively, the photochromic agent may be provided to a polymeric material following polymerization.

A photochromic agent may be incorporated into the polymeric material. The photochromic agent may be substantially contained within the polymer matrix and/or the interconnecting pores of the polymeric material. The photochromic agent may be substantially contained within the polymer matrix (or the interconnecting pores) due to hydrophobic/hydrophilic interactions, and/or due to the formation at least one bond between the photochromic agent and the polymer matrix. For example, in embodiments where the polymer matrix is substantially hydrophobic as compared to the interconnecting pores (e.g., comprising water), a hydrophobic photochromic agent may be substantially contained within the polymer matrix due to hydrophobic/hydrophilic interactions. It should be understood, however, that in some embodiments, the photochromic agent may be substantially contained in the interconnecting pores of the polymeric material. In such embodiments, the photochromic agent may not leach from the polymer matrix so long as some internal sections of the interconnecting pores or the surface openings are narrow such that the photochromic agent is substantially trapped inside these internal sections and may be retained during use. International Patent Application No. PCT/SG2009/000245, filed Jul. 9, 2009, entitled “Trapping Glucose Probe in Pores of Polymer,” published as WO/2010/005398 on Jan. 14, 2010, herein incorporated by reference, describes suitable methods and compositions for forming a polymeric material with selected pore size.

In some cases, a photochromic agent may be associated with a polymer matrix by the formation of at least one bond (e.g., covalent bond). In other instances, a least a portion of a photochromic agent may be cross-linked with a polymer matrix. As used herein, a component is “cross-linked” with a polymer matrix when the component comprises at least one bond (e.g., a covalent bond) to two or more adjacent chains of polymer. Those of ordinary skill in the art will be aware of methods for covalently linking a photochromic agent with the polymer matrix. For example, the photochromic group may be functionalized with at least one polymerizable group (e.g., a group that may be polymerized). The structure of the polymerizable group will depend on the structure of the polymer matrix being form. Non-limiting examples of polymerizable groups include p-vinylbenzene, a compound comprising an acrylate moiety (e.g., (methyl)acrylate), or the like. In some cases, a monomer may be functionalized with a photochromic group, and the ratio of the photochromic group in the polymer may be controlled by varying the ratio of unfunctionalized monomers to functionalized monomers. In such embodiments, the ratio of the unfunctionalized monomer to the functionalized monomer may be about 5:1, about 10:1, about 15:1, about 20:1, about 25:1, about 30:1, about 40:1, about 50:1, about 100:1, or greater, or may be between about 10:1 and about 100:1, or between about 20:1 and about 70:1, or between about 20:1 and about 40:1, or the like.

A photochromic agent may be any photochromic compound known to those of ordinary skill in the art. The term, “photochromic agent,” is given its ordinary meaning in the art and refers to any compound which exhibits a reversible change of color upon exposure to light. In some cases, the light is ultraviolet light. A photochromic agent may include the following classes of materials: chromenes (e.g., naphthopyrans, benzopyrans, indenonaphthopyrans, phenanthropyrans), spiropyrans (e.g., spiro(benzindoline) naphthopyrans, spiro(indoline)benzopyrans, spiro(indoline)naphthopyrans, spiro(indoline)quinopyrans, spiro(indoline)pyrans), oxazines (e.g., spiro(indoline)naphthoxazines, spiro(indoline)pyridobenzoxazines, spiro(benzindoline)pyridobenzoxazines, spiro(benzindoline)naphthoxazines, spiro(indoline)benzoxazines), mercury dithizonates, fulgides, fulgimides, or the like, or combinations thereof. In a particular embodiment, the photochromic agent is 6′-(2,3-dihydro-1H-indole-1-yl)-1,3-dihydro-3,3-dimethyl-1-propyl-spiro[2H-indole-2,3′-(3H)-naphtho(2,1-b)(1,4)oxazine, a spiro-naphthoxazine.

As used herein, a “photochromic amount” means an amount of a photochromic agent that is at least sufficient to produce a photochromic effect discernible to the naked eye upon activation. The concentration of photochromic agent in the polymerizable mixture may be selected based on a number of considerations such as the photochromic efficiency of the photochromic compound, the solubility of the photochromic compound (e.g., in the polymerizable material, in the fluid contained in the pores of the polymeric material, etc.), the thickness of the material or article (e.g., lens), and the desired darkness of the material or article (e.g., lens) when exposed to light. Typically, the more photochromic agent incorporated into an article, the greater the color intensity is up to a certain limit. Generally, there may be a point after which the addition of more photochromic agent does not have a noticeable effect. The polymerizable mixture or article may include more than one photochromic agent.

In addition, the concentration of the photochromic agent in the article or material may be varied in different locations of the article, as described herein. It will be appreciated that, by increasing the concentration of a photochromic agent in a given location of an article (e.g., lens), the percentage of light that is blocked at that location is generally increased as compared to locations where the concentration has not been increased. The term “photochromic region” refers to a portion of the article or material lens that includes one or more photochromic agents.

Photochromic materials for use with embodiments of the present invention generally at least partially block light in at least a portion of the visible spectrum (approximately 400 nm-700 nm). However, health advantages may be gained if a material blocks at least some light in either the ultraviolet or infrared portions of the spectrum.

In some embodiments, a photochromic material exhibits a color change upon exposure to any suitable light source which emits ultraviolet radiation. In one embodiment, the light source is sunlight. In another embodiment, the light source may be a mercury lamp or a xenon lamp. The exposure time required to exhibit a visible color change may vary depending upon various factors including, but not limited to, wavelength and/or intensity of the light.

Those of ordinary skill in the art will be aware of methods and techniques for determining both direct and reverse photochromism response times for a photochromic material. In some cases, the response time may be presented as a lifetime, T, where T may be calculated according to the following equation

T=1/k

where k is the rate constant. In some cases, the rate constant for reverse photochromism and/or direct photochromism of a material of the present invention is at least about 0.01 s⁻¹, about 0.02 s⁻¹, about 0.03 s⁻¹, about 0.05 s⁻¹, about 0.07 s⁻¹, about 0.10 s⁻¹, about 0.12 s⁻¹, about 0.15 s⁻¹, about 0.20 s⁻¹, about 0.3 s⁻¹, or greater. In some cases, the rate constant may be between about 0.01 s⁻¹ and about 0.4 s⁻¹, between about 0.02 s⁻¹ and about 0.25 s⁻¹, between about 0.05 s⁻¹ and about 0.15 s⁻¹, between about 0.08 s⁻¹ and about 0.12 s⁻¹, or the like.

Those of ordinary skill in the art will be aware of methods and techniques for preparing a microemulsion. The term “microemulsion,” is given its ordinary meaning in the art and refers to a thermodynamically stable dispersion of one liquid phase into another liquid phase. The microemulsion may be stabilized by an interfacial film of surfactant. Generally, one of the two liquid phases is hydrophilic or lipophobic (such as water) and the other is hydrophobic or lipophilic (such as oil). Typically, the droplet or domain diameters in microemulsions are about 100 nanometers or less, and thus the microemulsions are transparent (e.g., prior to a change in the color of a photochromic agent contained within the microemulsion). A microemulsion can be continuous or bicontinuous.

A microemulsion may be prepared, for example, by dispersing a mixture of components (e.g., monomer, surfactant, water) using standard techniques such as sonication, vortexing, or other agitation techniques for creating microdroplets of the different phases within the mixture. Alternatively, the mixture may be passed through a filter having pores on the nanometer scale so as to create fine droplets. Depending on the proportions of various components and the hydrophile-lipophile value of the surfactant, the droplets can be swollen with oil and dispersed in water (referred to as normal or O/W microemulsion), or swollen with water but dispersed in oil (referred to as inverse or W/O microemulsion), or the microemulsion can be bicontinuous.

As will be understood by one of ordinary skill in the art, a nanoporous and, in some cases, transparent polymer matrix may be obtained when the components of the microemulsion are in appropriate ratios and the droplets or domains have appropriate sizes. As is known to persons skilled in the art, to determine the appropriate proportions of the components suitable for forming a bicontinuous microemulsion, a ternary phase diagram for the monomer, water and the surfactant may be prepared. The region on the diagram corresponding to single-phase microemulsion may be identified and the proportions of the components may be so chosen such that they fall within the identified region. A person skilled in the art will be able to adjust the proportions according to the diagram in order to achieve a certain desirable property in the resulting polymeric material. Further, the formation of a bicontinuous microemulsion may be confirmed using techniques known to persons skilled in the art. For example, the conductivity of the mixture may increase substantially when the microemulsion is bicontinuous. The conductivity of the mixture may be measured using a conductivity meter after titrating a 0.1 M sodium chloride solution into the mixture. The water in the microemulsion can be pure water or a water-based liquid. The water may optionally contain various additives, as described herein.

Persons skilled in the art will understand how to combine different monomers and surfactants in different ratios to achieve the desired effect on the various properties of the resulting polymeric material, for example to improve the mechanical strength or hydrophilicity of the resulting polymeric material. Generally, the choice and weight ratio of the particular components (e.g., monomer and surfactant) depends on the application of the resulting polymeric material. The ratios may be selected such that the resulting polymeric material is suitable and compatible with the environment in which the polymeric material is to be used and has the desired properties. In some cases, the water content in the bicontinuous emulsion is between about 10% to about 50%, between about 15% and about 45%, between about 15% and about 40%, between about 20% and about 35%, or between about 20% and about 30%. The surfactant may be present in an amount between about 10% and about 50%, between about 15% and about 45%, between about 20% and about 40%, between about 30% and about 50%, between about 10% and about 30%, between about 20% and about 30%, or between about 15% and about 25%. The one or more monomers may be present in an amount between about 20% and about 70%, between about 30% and about 70%, between about 40% and about 70%, between about 40% and about 60%, or the like. A cross-linker may be present in an amount between about 0.1% and about 10%, between about 1% and about 10%, between about 5% and about 10%, between about 5% and about 15%, between about 3% and about 8%, between about 0.1% and about 5%, between about 0.1% and about 3%, between about 0.5% and about 2%, between about 0.5% and about 1.5%, or about 1.0% about 2%, about 3%, about 4%, about 5%, about 6%, about 7%, about 8%, about 9%, about 10%, or more. The photochromic agent may be present in an amount between about 0.01% and about 5%, between about 0.1% and about 5%, between about 0.01% and about 3%, between about 0.01% and about 2%, between about 0.01% and about 1%, between about 0.01% and about 0.5%, between about 0.01% and about 0.2%, or about 0.1%. The emulsion may additionally comprise one or more additives (e.g., including a polymerization initiator, as described herein) in an amount between 0.01% and about 5%, between about 0.1% and about 5%, between about 0.01% and about 3%, between about 0.01% and about 2%, between about 0.01% and about 1%, between about 0.01% and about 0.5%, between about 0.01% and about 0.2%, or about 0.1%, about 0.5%, about 1.0%, about 2.0%, about 2.5%, about 3.0%, about 4.0%, about 0.5%, or more. In a particular embodiment, a bicontinuous microemulsion comprises from about 15 to about 50% for water, from about 5% to about 40% for the monomer, and from about 10% to about 50% for the surfactant.

In some embodiments, the water content of a polymeric material may determined as the equilibrium water content. Those of ordinary skill in the art will be aware of methods for determining an equilibrium water content. The equilibrium water content (Q) of a polymeric material may be calculated as follows:

Q=(W_{S}−W_{d})×100/W_{S}, (1)

where W_{S} is the saturation weight and W_{d} is the dry weight. The saturation weight may be measured after immersing the polymeric material in water for a period of time so that the total weight will no longer increase significantly upon further immersion.

Various other measurable factors may be determined and/or tuned for the polymeric material by varying the ratio of components. In some cases, the polymeric material may have a light transmission percentage higher than about 80%, about 85%, about 88%, about 90%, about 92%, about 95%, or greater, or between about 80% and about 100%, between about 80% and about 95%, between about 85% and about 95%, or between about 88% and about 93%. The refractive index of the polymeric material may be about 0.7, about 0.8, about 0.9, about 1.0, about 1.1, about 1.2, about 1.3, about 1.4, about 1.5, or greater, or between about 0.5 and about 1.5, between about 0.7 and about 1.4, or between about 0.9 and about 1.3. The polymeric material may have a glucose diffusion permeability coefficient of at least about 1×10⁻⁶ cm²/s, about 2×10⁻⁶ cm⁻²/s, about 3×10⁻⁶ cm⁻²/s, about 4×10⁻⁶ cm⁻²/s, about 5×10⁻⁶ cm⁻²/s, or greater, or between about 2.1×10⁻⁶ cm⁻²/s to about 3.2×10⁻⁶ cm⁻²/s, between about 1×10⁻⁶ cm⁻²/s to about 5×10⁻⁶ cm²/s, between about 2×10⁻⁶ cm⁻²/s to about 4×10⁻⁶ cm²/s, between about 1×10⁻⁶ cm⁻²/s to about 7×10⁻⁶ cm⁻²/s, or between about 0.1×10⁻⁶ cm⁻²/s to about 10×10⁻⁶ cm⁻²/s. The polymeric material may have an albumin diffusion permeability coefficient of at least about 1.0×10⁻⁷ cm⁻²/s, about 1.2×10⁻⁷ cm²/s, about 1.4×10⁻⁷ cm⁻²/s, about 1.6×10⁻⁷ cm⁻²/s, about 1.8×10⁻⁷ cm⁻²/s, about 2.0×10⁻⁷ cm⁻²/s, or greater, or between about 1.4×10⁻⁷ cm⁻²/s and about 1.8×10⁻⁷ cm⁻²/s, between about 1.0×10⁻⁷ cm⁻²/s and about 2.0×10⁻⁷ cm⁻²/s, between about 1.2×10⁻⁷ cm⁻²/s and about 2.0×10⁻⁷ cm⁻²/s, between about 1.4×10⁻⁷ cm⁻²/s and about 2.0×10⁻⁷ cm⁻²/s, between about 1.4×10 cm⁻²/s and about 1.6×10⁻⁷ cm⁻²/s. The polymeric material may have a tensile strength of at least about 1 MPa, about 1.5 MPa, about 2 MPa, about 2.5 MPa, about 3 MPa, about 4 MPa, or greater, or between about 1 MPa and about 10 MPA, between about 1 MPa and about 5 MPA, between about 2 MPa and about 5 MPA, between about 1 MPa and about 3 MPA, or between about 2 MPa and about 4 MPA. The polymeric material may have a Young's modulus of at least about 60 MPa, about 80 MPa, about 90 MPa, about 100 MPa, about 110 MPa, or greater, or between about 50 MPa and about 150 MPa, between about 60 MPa and about 140 MPa, between about 80 MPa and about 120 MPa, or between about 60 MPa and about 110 MPa. Those of ordinary skill in the art will be aware of methods and devices for determining the above parameters for a polymeric material. For example, oxygen permeability may be measured using a Polarographic method, also known as the FATT method named after Dr. Irving Fatt. This method may be performed with a Model 201T Oxygen Permeometer, available from Rehder™, M201T.

For medical applications, the polymeric material should be safe and biocompatible with human cells. For use as contact lenses, it is desirable that the polymeric material is permeable to fluids such as gases (e.g., O₂ and CO₂), various salts, nutrients, water and diverse other components of the tear fluid. The presence of interconnecting pores distributed throughout the polymeric material may facilitate the transport of gases, molecules, nutrients, and/or minerals through the eye and the surroundings.

The interconnecting pores of the polymeric material may have a pore diameter of between about 10 nm and about 100 nm, between about 20 nm and about 90 nm, or between about 30 and about 80 nm. The pores may have round or other cross-sectional shapes and may have different sizes. As used herein, a pore diameter refers to the average or effective diameter of the cross-sections of the pores. The effective diameter of a cross-section that is not circular equals the diameter of a circular cross-section that has the same cross-sectional area as that of the non-circular cross-section. In some embodiments, such as when the polymeric material is swellable when the pores are filled with water, the sizes of the pores may change depending on the water content in the polymeric material. When the polymeric material is dried, some or all of the pores may be filled or partially filled by a gas such as air. The polymeric material may thus behave like a sponge. In alternative embodiments, the pore diameter may be in the range from about 10 nm to 100 nm when the polymeric material is in a dry condition wherein the water content of the polymeric material is at or near minimum.

The pores may be randomly distributed. Some of the pores may be closed pores, meaning that they are not connected or joined with other pores or open to the surfaces of the polymeric material. It is not necessary that all of the pores are interconnected. Depending on use, the polymeric material can be prepared to have more or less interconnected pores as would be understood by an ordinary person skilled in the art.

In some cases, the polymeric material is substantially transparent (e.g., prior to a color change in the photochromic material). As used herein, the term “transparent” broadly describes the degree of transparency that is acceptable for a contact lens or like devices, for example the degree of transmission of visible light through the polymeric material equivalent to that of other materials employed in the manufacture of contact lenses or other ophthalmic devices.

A bicontinuous microemulsion may be polymerized by standard techniques known to those of ordinary skill in the art. For example, it may be polymerized by heat, the addition of a catalyst, by irradiation of the microemulsion or by introduction of free radicals into the microemulsion. The method of polymerization chosen will be dependent on the nature of the components of the microemulsion.

The monomers for forming bicontinuous microemulsion can be any suitable monomer known to those of ordinary skill in the art, which is capable of copolymerizing with another monomer (e.g., a surfactant) to form a polymeric material. While the monomer is copolymerizable with another monomer such as the surfactant, the monomer may also be polymerizable with itself. The type and amount of the monomer that may be employed to prepare a suitable bicontinuous microemulsion will be known to a person of ordinary skill in the art. Exemplary monomers are ethylenically unsaturated monomers including methyl methacrylate (MMA), 2-hydroxylethyl methacrylate (HEMA), 2-hydroxylethyl acrylate, monocarboxylic acids such as acrylic acid (AA) and methacrylic acid (MA), glycidyl methacrylate (GMA), and silicone-type monomers, or the like. Suitable combinations of these monomers can also be used.

In some embodiments, more than one monomer may be provided. In some cases, a combination of monomers comprises a first monomer more hydrophilic than 2-hydroxyethyl methacrylate (HEMA), and a second monomer as hydrophilic as, or less hydrophilic than, HEMA. The monomers in the bicontinuous microemulsion may be polymerized to form a porous polymeric material. In exemplary embodiments of the present invention, the combination of the first and second monomers and their concentrations may be conveniently selected so that the resulting polymeric material has the desired properties for a particular application.

In some cases, the first monomer may comprise N-vinylpyrrolidone (NVP) or methacrylic acid (MAA) and the second monomer may comprise HEMA or methyl methacrylate (MMA) 2-hydroxylethyl acrylate, monocarboxylic acids, glycidyl methacrylate (GMA), and silicone-based monomers. In other embodiments, NVP or MAA may be replaced with one or more other highly hydrophilic monomers. A monomer is considered to be “highly” hydrophilic herein when it is more hydrophilic than HEMA. Typically, the more hydrophilic terminal groups a monomer has, the more hydrophilic the monomer. Thus, a highly hydrophilic monomer can have more hydrophilic terminal groups in its base structure than HEMA does. Alternatively, the hydrophilic groups in a highly hydrophilic monomer may be individually more hydrophilic than the hydrophilic groups of HEMA. The hydrophilicity of a material may be measured by its equilibrium water content. As can be appreciated, NVP and MAA are highly hydrophilic. There are other materials, such as silicone-based monomers, which are also highly hydrophilic. NVP and MA may thus be replaced by such other materials. A highly hydrophilic material may be amphiphilic. International Patent Application No. PCT/SG2009/000097, filed Mar. 19, 2009, entitled “Forming Copolymer from Bicontinuous Microemulsion Comprising Monomers of Different Hydrophilicity,” published as WO/2010/107390 on Sep. 23, 2010, herein incorporated by reference, describes suitable combinations of monomers and methods for forming a polymeric material.

As will be understood by those of ordinary skill in the art, a polymerizable surfactant may be capable of polymerizing with itself and/or with other monomeric compounds to form a polymeric material. The surfactant for the mixture can be any suitable surfactant that can co-polymerize with at least one of the monomers in the microemulsion. As can be appreciated, when the surfactant is copolymerized into the polymeric material, there is no need to separate the surfactant from the polymeric material after polymerization. This can be advantageous as the polymeric material formation process is simplified. The surfactant can be anionic, non-ionic, or zwitterionic. In a particular embodiment, the surfactant is non-ionic. Exemplary surfactants include poly(ethylene oxide)-macromonomer (PEO-macromonomer), such as ω-methoxy poly(ethylene oxide)₄₀ undecyl α-methacrylate macromonomer denoted herein as C₁-PEO-C₁₁-MA-40. The chain length of the macromonomer can be varied. For example, the macromonomer may be in the form of R¹O(CH₂CH₂O)ₙ—(CH₂)ₙV, where R¹ is either hydrogen or an alkyl group (e.g., C₁-C₅ alkyl), n is an integer between about 5 and about 200, or between about 10 and about 110, and V is a polymerizable group. The structure of the polymerizable group will depend on the type of polymer matrix being formed. In some cases, the polymerizable group is p-vinylbenzene, a compound comprising an acrylate moiety (e.g., (methyl)acrylate), or the like. In some embodiments, the surfactant may be a zwitterionic surfactant such as {SO₃⁻}{(CH₂)ₘ⁺}NCHCHCHN(CH₂)ₚV, where m is an integer ranging from 1 to 20, n is an integer ranging from 6 to 20, p is an integer ranging from 10 to 110, and V is copolymerizable group.

Polymerization of the microemulsion may involve the use of a catalyst. The catalyst may be any catalyst or polymerization initiator that promotes polymerization of the monomers and the surfactant. The specific catalyst chosen may depend on the particular monomers, and polymerizable surfactant used or the method of polymerization. For example, polymerization can be achieved by subjecting the microemulsion to ultraviolet (UV) radiation if a photo-initiator is used as a catalyst. Exemplary photo-initiators include 2,2-dimethoxy-2-phenyl acetophenone (DMPA) and dibenzylketone. A redox-initiator may also be used. Exemplary redox-initiators include ammonium persulphate and N,N,N′,N′-tetramethylethylene diamine (TMEDA). A combination of photo-initiator and redox-initiator may also be used. In this regard, including in the mixture an initiator can be advantageous. The polymerization initiator may be present in an amount between about 0.1% and about 5%, between about 1% and about 5%, between about 0.1% and about 4%, between about 0.1% and about 3%, between about 0.1% and about 1%, between about 2% and about 4%, between about 0.1% and about 0.5%, or between about 0.1 wt % to about 0.4 wt % of the microemulsion.

To promote cross-linking between polymer molecules in the resulting polymeric material, a cross-linker may be added to the mixture. Suitable cross-linkers include ethylene glycol dimethacrylate (EGDMA), diethylene glycol dimethacrylate, diethylene glycol diacrylate, or the like. As can be understood, the more the polymer molecules are cross-linked, the more difficult it for an additive (e.g., drug) or photochromic agent to diffuse or migrate through the polymeric material, thereby resulting in a slower release of the additive or photochromic agent. The content of the cross-linker can therefore be selected to adjust the release rate. Increasing the overall concentration of the cross-linker can also improve the mechanical strength of the resulting polymeric material.

The microemulsion may be formed into a desired end shape and size prior to polymerization. For example, a sheet material may be formed by pouring or spreading the mixture into a layer of a desired thickness or by placing the mixture between glass plates prior to polymerization. The mixture may also be formed into a desired shape such as a rod, for example, by pouring the mixture into a mold or cast prior to polymerizing. In some cases, however, following polymerization, the polymeric material may be formed into a desired end shape using cutting techniques (e.g., using lasers). In some cases, the microemulsion may be stored (e.g., at low temperatures) for a period of time, prior to forming the desired end-shape article and/or prior to polymerization. For example, the microemulsion may be stored at a suitable temperature (e.g., about 0° C., about 2° C., about 4° C., about 6° C., about 8° C., etc.) for at least about 1 day, about 2 days, about 3 days, about 4 days, about 1 week, about 1 month, prior to forming the desired end-shape article, with essentially no substantial changes in the end properties of the end-shape article as compared to an article formed of the material at a time point essentially immediately after forming the end-shape article.

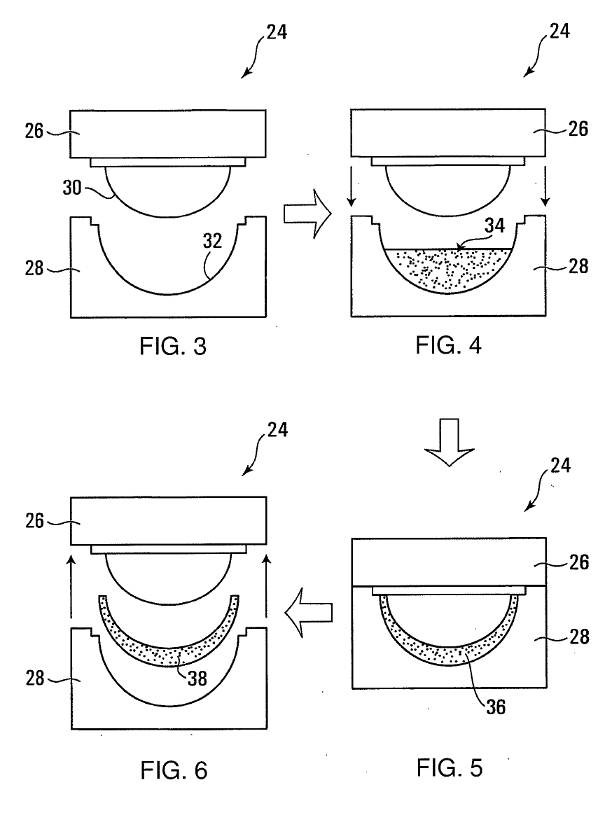

In some embodiments, a microemulsion may be formed into a desired end shape and size prior to polymerization. In one embodiment, contact lens 10 may be formed from the microemulsion according to the process illustrated in FIGS. 3 to 6. As shown in FIG. 3, a mold 24 is provided, which includes a male portion 26 and a female portion 28. Male and female portions 26 and 28 can be detachably coupled. The inner surface 30 of male portion 26 is convex shaped and the inner surface 32 of female portion 28 is correspondingly concave shaped so that when the male and female portions are coupled together the inner surfaces 30 and 32 define a desired profile for the content lens. As shown in FIG. 4, a suitable amount of the prepared microemulsion 34 is first deposited into female portion 28. Male portion 26 is then coupled to female portion 28 to compress microemulsion 34 into the desired shape 36 defined by inner surfaces 30 and 32, as shown in FIG. 5. Alternatively, male and female portions 26 and 28 may be first coupled and the microemulsion may be then injected into the cavity of the mold. For this purpose, an injection port (not shown) may be provided. Microemulsion 36 in mold 24 is then subject to polymerization reactions. Polymerization may be effected by irradiation such as ultraviolet (UV) irradiation. The monomers are then polymerized to form a polymeric material as described above. As shown in FIG. 6, the resulting polymeric material forms a contact lens 38 which has the desired shape. Contact lens 38 may be removed from mold 24 after polymerization.

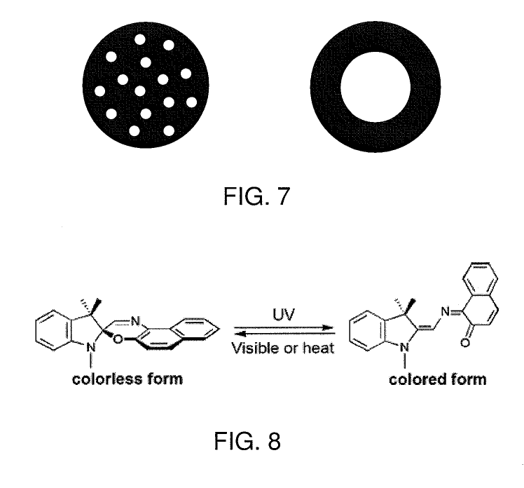

The polymeric materials may also be used, in some cases, for the preparation of pinhole lenses. Pinhole lenses will be known to those of ordinary skill in the art. These lenses utilize theories of pinhole imaging, commonly understood in optics as a method to reduce geometrical aberrations, e.g., astigmatism, spherical aberration, and coma. By restricting a person's vision to a small “pinhole” aperture, visual deficiencies are greatly reduced, or even effectively removed. Non-limiting types of pinhole lenses are shown in FIG. 7. Those of ordinary skill in the art will be aware of techniques for forming a pinhole lens. For example, in some cases, a portion of a mold may comprise a bicontinuous emulsion comprising a photochromic agent and another portion of the mold may comprise a bicontinuous emulsion not comprising a photochromic agent. The bicontinuous emulsion may then be polymerized, thus forming an article comprising at least one region comprising a photochromic agent and at least one other region not comprising a photochromic agent. Another method for forming a pinhole lens comprising forming a first lens comprising a photochromic agent and a second lens not comprising the photochromic agent. The two lenses may be stacked or otherwise associated with each other and a portion of the photochromic lens may be removed, thereby forming a pinhole lens. In some cases, lasers may be used to cut and/or remove one or more portions of a photochromic lens. In yet another example, at least a portion of a material not comprising the photochromic material may be dipped and/or coated with a photochromic polymeric material.

After polymerization, the polymeric material may be rinsed and/or equilibrated with water to remove un-reacted monomers and/or other components that have not been incorporated into the polymeric material. In some cases, a small percentage of the additives incorporated in the polymeric material may be lost during rinsing but the amount lost can be limited by controlling the duration of rinsing. A rinsed polymeric material may be optionally dried and sterilized in preparation for use in a medical or clinical application. Both drying and sterilization can be accomplished in any suitable manner, which is known to person of skill in the art. In some embodiments, both drying and sterilization can be affected at a low temperature so as not to adversely affect the additive or photochromic agent, for example using ethyleneoxide gas or UV radiation.

In some embodiments, the polymeric material may comprise one or more additives. In some cases, an additive may be sufficiently contained within the polymer matrix, of the water in the interconnected pores, or both. Such additives can be selected for achieving one or more desired properties in the resulting polymeric material, and can include one or more of a drug, a protein, an enzyme, a filler, a dye, an inorganic electrolyte, a pH adjuster, or the like.

In some embodiments, a drug such as an ophthalmic drug may be incorporated into the microemulsion. The drug may be dispersed in the aqueous domains or in the oil domains of the microemulsion, or in both domains including at the interface of the two domains. When the drug is initially dispersed in the oil domains, it is likely dispersed in the polymer matrix after polymerization. When the drug is initially dispersed in the aqueous domains, it is likely dispersed in the water in the pores after polymerization. Drugs that can be incorporated in the polymeric material can vary and can be either hydrophilic or hydrophobic, water soluble or water insoluble. Those of ordinary skill in the art will understand how different drugs will be dispersed in the microemulsion depending on their properties such as hydrophilicity or lipophilicity. International Patent Application No. PCT/SG2004/000237, filed Aug. 3, 2004, entitled “Polymer having Interconnected Pores for Drug Delivery and Method,” published as WO 2006/014138 on Feb. 9, 2006, herein incorporated by reference, describes suitable drugs, and methods for incorporating a drug into a polymer matrix.

Non-limiting examples of ophthalmic drugs include anti-glaucoma agents such as a beta adrenergic receptor antagonist, e.g., timolol maleate, and other therapeutic agents such as antibiotic agents, antibacterial agents, anti-inflammatory agents, anaesthetic agents, anti-allergic agents, polypeptides and protein groups, lubricating agents, any combination or mixture of the above, and the like.

The amount of the drug to be included may be determined based on various factors. In general, the drug should have a concentration suitable for providing the desired therapeutic dosage, as would be known in the art. For ophthalmic drug delivery, the transparency and clarity of the resulting polymeric material is one of the factors may depend on the concentration of the drug in the polymeric material.

In some cases, a polymeric material may comprise a glucose probe. A glucose probe can be any compound that generates a detectable spectral signal, such as a change in fluorescence response, in the presence of glucose. For instance, the glucose probe may react with glucose on contact, thus forming a new compound structure which has a fluorescence spectrum different from that of the original probe molecule. A glucose probe may be trapped in the interconnecting pores of a polymer material and/or with bond the probe to the polymer. When internal pores in the polymer are connected with one another and to surface pores, glucose may travel through the connected pores to interact with the probe in the pores during use. To prevent leaching of the probe, the pores can be connected through openings sized to restrict passage of the probe through the openings. A suitable glucose probe may be a boronic acid probe, such as a boronic acid-based fluorophore. For example, a boronic acid may be used. The boronic acid may have the formula of R—B(OH)₂, where R is alkyl, alkenyl, cycloalkyl, cycloalkenyl, alkoxyalkyl, alkoxyalkenyl, or aryl arylakyl. Suitable boronic acids include 1,3-diphenylprop-2-en-1-one, or alternatively expressed as 3-[4′(dimethylamino)phenyl]-1-(4″-boronophenyl)-prop-2-en-1-one; and 1,5-diphenylpenta-2,4-dien-1-one, alternatively expressed as 5-[4″-(dimethylamino)phenyl]-1-(4′-boronophenyl)-pent-2,4-dien-1-one. International Patent Application No. PCT/SG2009/000245, filed Jul. 9, 2009, entitled “Trapping Glucose Probe in Pores of Polymer,” published as WO/2010/005398 on Jan. 14, 2010, herein incorporated by reference, describes suitable glucose probes and methods for containing a glucose probe in a polymeric material.

In some embodiments, a polymeric material may comprise a wetting agent. A wetting agent may be any suitable wetting agent, subject to constraints in any given particular application. For example, for contact lens applications, the wetting agent should be compatible with human eye. In a contact lens application, suitable wetting agents may include hyaluronic acid (HA), acrylated HA (AHA), methacrylated hyaluronic acid (MeHA), polyvinylpyrrolidone (PVP), dextran, or other wetting agents that are suitable for ophthalmic applications. In some applications, wetting agents such as carboxymethylcellulose (CMC), hydroxypropyl methylcellulose (HPMC), glycerine, chitosan, polyvinylalcohol, or the like, may be suitable.

Hyaluronic acid may also be referred to as hyaluronate or hyaluronan. A hyaluronic acid is a glycosaminoglycan, also called mucopolysaccharide, which is a polymer of disaccharides, composed of D-glucuronic acid and D-N-acetylglucosamine, linked together via alternating β-1,4 and β-1,3 glycosidic bonds. An exemplary hyaluronic acid A is a sodium hyaluronate. In some cases, a wetting agent may be cross-linked with the polymer matrix. International Patent Application No. PCT/SG2007/000398, filed Nov. 17, 2007, entitled “Porous Polymeric Material with Cross-Linkable Wetting Agent,” published as WO 2008/060249 on May 22, 2008, herein incorporated by reference, describes suitable wetting agents including wetting agents for cross-linking with the polymer matrix, and methods for incorporating wetting agents into a polymer matrix.

Conveniently, the polymeric materials according to various embodiments of the invention can be made compatible with human dermal fibroblasts cells and mechanically strong and can be advantageously used to manufacture contact lenses for placement on the eye.

The polymeric materials described above are useful not only for contact lens applications, but also useful in other applications. For example, the exemplary materials and processes described herein, or similar materials or processes, may be utilized to prepare hydrophilic, nanoporous materials for use in applications such as prescription lenses, 3-D (dimensional) tissue engineering scaffolds, artificial cornea, or the like. Since only one polymerization step is required, in some cases, to prepare the polymeric material incorporating a photochromic material, the process can be simple and inexpensive.

In some embodiments, the present invention provides an optical device comprising a photochromic agent rendering the device switchable from a first, relatively transparent state to a second, at least partially opaque state, whereby transmission of visible light through the optical pathway can change by at least about 20%, at least about 30%, at least about 40%, at least about 50%, at least about 60%, at least about 70%, at least about 80%, or more, upon switching of the device from the first state to the second state, and from the second state to the first state, each within a period of time of no more than about 1 second, about 5 seconds, about 10 seconds, about 15 seconds, about 20 seconds, about 25 seconds, about 30 seconds, about 35 seconds, about 40 seconds, about 50 seconds, about 60 seconds, about 90 seconds, about 120 seconds, or the like upon exposure to appropriate electromagnetic radiation and/or thermal relaxation. The optical device may have an optical pathway through the device with a maximum length of less than about 0.5 mm, about 1 mm, about 1.5 mm, about 2 mm, about 2.5 mm, about 3 mm, about 4 mm, about 5 mm, or the like. The optical device may be a contact lens.

In some embodiments, a composition comprises between about 15%, and about 25% ω-methoxy poly(ethylene oxide)₄₀ undecyl α-methacrylate macromonomer (PEO-R-MA-40), between about 15% and about 20% glycidyl methacrylate (GMA), between about 30% and about 50% 2-hydroxyethyl methacrylate (HEMA), between about 15% and about 25% water, between about 0.1% and about 10% ethyleneglycol dimethacrylate (EGDMA), between about 0.1% and about 5% 2,2-azobis[2-(2-imidazolin-2-yl)propane]dihydrochloride (AIPH), and between about 0.05% and about 5% 6′-(2,3-dihydro-1H-indole-1-yl)-1,3-dihydro-3,3-dimethyl-1-propyl-spiro[2H-indole-2,3′-(3H)-naphtho(2,1-b)(1,4)oxazine (SPO). In a particular embodiment, a composition comprises about 20% PEO-R-MA-40, about 17% GMA, about 43% HEMA, about 20% water, about 1.0% EGDMA, about 0.3% AIPH, and about 0.1% SPO. In another particular embodiment, a composition comprises about 18.2% PEO-R-MA-40, about 14.1% GMA, about 37.9% HEMA, about 18.2% water, about 8.8% EGDMA, about 2.7% AIPH, and about 0.1% SPO.

A “polymer,” as used herein, is given its ordinary meaning as used in the art, i.e., a molecular structure comprising one or more repeat units (monomers), connected by covalent bonds. The repeat units may all be identical, or in some cases, there may be more than one type of repeat unit present within the polymer.

The terms “hydrophobic” and “hydrophilic” are given their ordinary meaning in the art and, as will be understood by those of ordinary skill in the art, in many instances herein, these are relative terms. Although specific parameters or limitations on the meaning of a “hydrophobic material” (e.g., polymer matrix) would be inappropriate given different relative hydrophobicities, in general, a hydrophobic polymer matrix is one that, when formed into a material suitable for a contact angle measurement, will result in a water contact angle of greater than about 50°.

As used herein, “substantially,” in connection with a component (e.g., a photochromic agent) being contained within a polymer matrix (or interconnecting cores), means that at least about 25%, at least about 35%, at least about 50%, at least about 60%, at least about 75%, at least about 85%, or at least about 90%, at least about 95%, or more, of the component is encapsulated in and/or compounded with the polymeric material.

As used herein, a “subject” or a “patient” refers to any mammal (e.g., a human). Examples include a human, a non-human primate, a cow, a horse, a pig, a sheep, a goat, a dog, a cat, or a rodent such as a mouse, a rat, a hamster, or a guinea pig. Generally, or course, the invention is directed toward use with humans. A subject may be a subject needing corrective lenses.

These polymeric materials can have various desirable physical, chemical, and biochemical properties. To illustrate, the preparation and properties of sample polymeric materials are described below. The following examples are intended to illustrate certain embodiments of the present invention, but do not exemplify the full scope of the invention.

### Example 1

The following describes the preparation and characterization of a non-limiting photochromic polymeric material of the present invention.

The photochromic agent used in this example is 6′-(2,3-dihydro-1H-indole-1-yl)-1,3-dihydro-3,3-dimethyl-1-propyl-spiro[2H-indole-2,3′-(3H)-naphtho(2,1-b)(1,4)oxazine, a spiro-naphthoxazine (SPO). The structure of SPO and one of its open forms is shown in FIG. 8 (note: only one of the several colored forms is represented in the scheme). Upon irradiation with ultraviolet (UV) light, the colorless SPO undergoes a heterolytic cleavage of the spiro C—O bond in the oxazine ring, resulting in the colored form of photomerocyanine (PMC), which then reverts back to SPO either thermally or upon irradiation with visible light. The open structure is best described in the quinoidal form for the PMC dye. As described herein, SPO was incorporated in a disposable lens system made by bicontinuous microemulsion with different aqueous contents; such a polymeric material could improve the discoloration characteristics and reduce the discoloration time. The photochromic dye was incorporated predominantly within the hydrophobic domains of the nanostructure, and exhibited direct photochromism with very rapid response times. The fits for decay time were mono-exponential for the dye, indicating a homogeneous dye environment. Moreover, the occluded dyes exhibited direct photochromism even after months. The SPO-doped lenses further showed a slightly faster response when they were completely dried. These findings were important for the ophthalmic applications of photochromic microemulsion-derived lenses.

The nanostructured lenses were typically prepared by polymerizing the bicontinuous microemulsion precursor derived via the self-assembly of amphiphilic templates that consisted of ω-methoxy poly(ethylene oxide)₄₀ undecyl α-methacrylate macromonomer (C₁-PEO-C₁₁-MA-40 or PEO-macromonomer), 2-hydroxyethyl methacrylate (HEMA), glycidyl methacrylate (GMA) and water. The resulting bicontinuous phase offered exceptional control over the nanostructure, yielding an architecture that was well-suited for the incorporation of photochromic dye. The compositions of selected microemulsions are listed in Table 1. The resulting lenses were molded using a mold-casting technique. They lenses were substantially transparent and showed improved hydrophilicity and oxygen permeability (Dₖ) (from 13 to 21) with increasing water content. The Young's modulus varied from 0.44 to 0.63 MPa with decreasing water content. No significant amount of the tensile strength was lost when the water content of these materials was further increased to 50-60 wt %. The Young's modulus and tensile strength (0.20-0.26 MPa) suggested that these materials were sufficiently durable for contact lens applications (see Table 1).

TABLE 1
Synthesis and characteristics of SPO-doped microemulsion-derived
nanostructured polymers.

| | PEO-20 | PEO-25 | PEO-30 |
|---|---|---|---|
| Composition of bicontinuous | | | |
| microemulsion (wt %) | | | |
| PEO-R-MA-40 | 20.0 | 20.0 | 20.0 |
| GMA | 17.0 | 15.0 | 13.0 |
| HEMA | 43.0 | 40.0 | 36.0 |
| Water | 20.0 | 25.0 | 30.0 |
| Ethyleneglycol dimethacrylate (EGDMA) | 1.0 | 1.0 | 1.0 |
| 2,2-azobis[2-(2-imidazolin-2- | 0.3 | 0.3 | 0.3 |
| yl)propane]dihydrochloride (AIPH) | | | |
| SPO | 0.1 | 0.1 | 0.1 |
| Materials Characteristics | | | |
| % of light transmission (400-800 nm) | 91 | 91 | 92 |
| Refractive index | 1.3 | 1.4 | 1.4 |
| Water content in polymer (wt %) | 46 | 50 | 50 |
| Oxygen permeability | 13 | 16 | 21 |
| Tensile strength (MPa) | 0.26 | 0.22 | 0.20 |
| Young's modulus (MPa) | 0.63 | 0.52 | 0.44 |

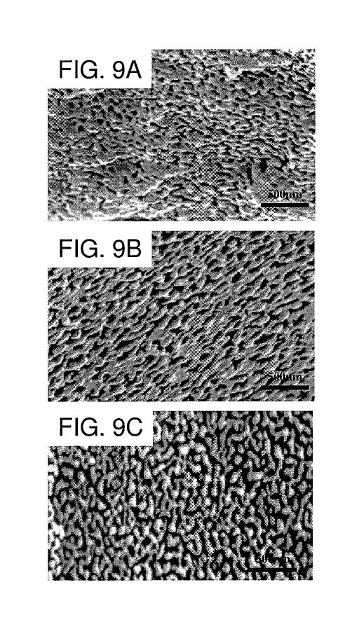

Time-dependent response curves were obtained by following the signal at the peak wavelength in the transient absorption spectra. Spectra of solutions (not shown) and as-synthesized lenses were obtained with an Agilent 5453 UV-visible spectrophotometer. The solutions from which the lenses were prepared were colorless. The lenses were completely transparent in the visible range, with transmissions of ˜97% indicating that the dye was present in their closed form. SEM micrographs (FIG. 9) showed the continuity of the bicontinuous microemulsion. Specifically, FIG. 9 shows FESEM micrographs of the fractured cross-sections of nanostructured lenses: (a) PEO-20, (b) PEO-25, and (c) PEO-30. The black and white stripes represent water channels and the polymer matrix domains, respectively. The winding dark strips in the SEM micrographs represented the aqueous channels, while the white domains represented the polymer matrix. Both the aqueous and polymer matrix domains were randomly distributed in the polymerized microemulsions. When the water content was increased to 30 wt %, more interwining and wider aqueous channels were formed. The nanometer-scaled separation of organic and aqueous domains constituted the biphasic nature of the nanostructure. The chemical surrounding of the dye species could therefore be tuned by the composition of the bicontinuous microemulsion.

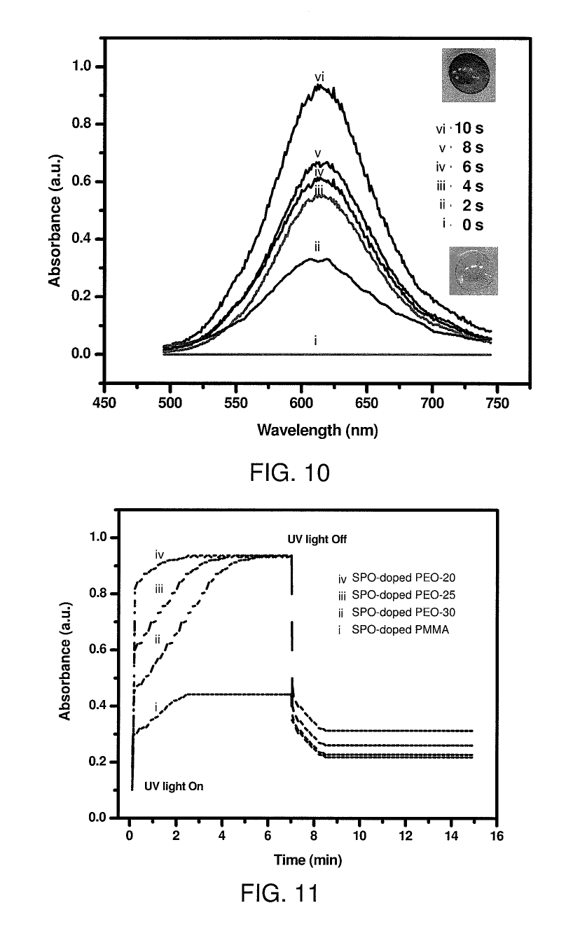

The transmission of the dye-doped lenses did not decrease after several weeks. Typical spectra of the lenses during UV exposure are depicted in FIG. 10. The SPO-doped lens showed only one broad absorption in the visible range with a maximum at 620 nm (PEO-20), and varied as the microemulsion water content was increased from 20 to 30 wt % (not shown). Visual inspection of the coloration/decoloration kinetics with UV irradiation of the SPO-doped lenses showed that the dyes exhibited similar decoloration kinetics as SPO-doped liquid precursor. The SPO-doped nanostructured lenses underwent rapid thermal fading, becoming colorless again within a few seconds after the removal of UV light. The optical intensity at 620 nm exponentially increased with increasing UV irradiation time at a given wavelength range of light as shown in FIG. 10. FIG. 10 shows changes in the absorbance spectra of the nanostructured PEO-20 lens doped with 0.1 wt % SPO upon UV irradiation at 365 nm for 2, 4, 6, 8, and 10 min. The changes in absorbance spectra indicated that the oxazine ring was opened by light irradiation, as illustrated by the absorption band at 620 nm. The opened and closed structures of SPO associated with the photochromism at different wavelength ranges were illustrated schematically in FIG. 8. The normal and reverse photochromism of SPO, i.e. conversion of the colorless form to the colored form upon irradiation and vice versa, were observed. The exponentially increasing absorbance at 620 nm during photoirradiation indicated the presence of an exponentially increasing fraction of the opened form of SPO.

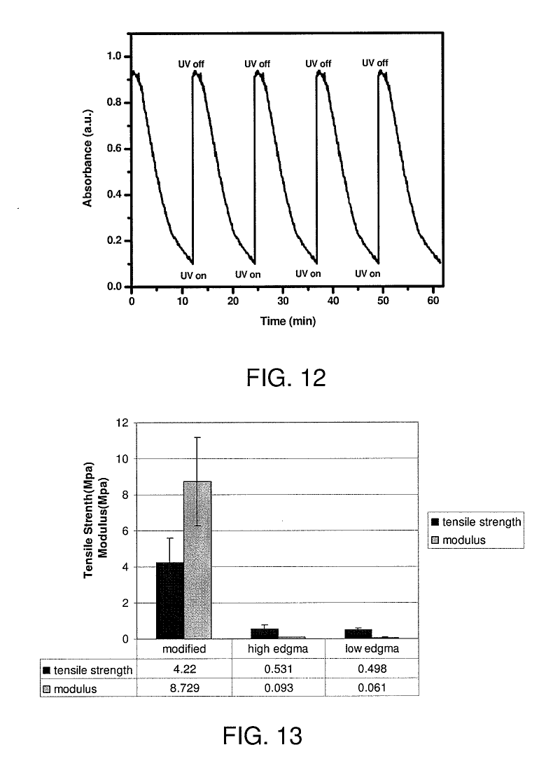

As previously stated, SPO photochromics have important applications in ophthalmic lenses due to their dark coloration, large extinction coefficients in the open form, inherent fatigue resistance, and moderately good switching performance. In testing the switching performance, the coloration of the lenses prepared with different water contents was found to be approximate 2 times greater than that of the control (PMMA) after 5 min of irradiation (FIG. 11). Specifically, FIG. 11 shows the absorbance as a function of time for the coloration and decoloration of SPO-doped nanostructured PEO lenses and control (SPO in a rigid polymer host matrix PMMA). SPO concentration=0.1 wt %. Absorbance was monitored at iₘₐₓ of the colored form of SPO (620 nm). A typical set of time-dependent spectral data for several coloration/decoloration cycles for PEO-20 is given in FIG. 12. Specifically, FIG. 12 shows the time-dependent photocoloration and bleaching for SPO-doped nanostructured PEO-20 lens. No photodecomposition was observed over multiple exposure cycles. From the bleaching curves, a rate constant k=0.10 s⁻¹ was determined for a sample that was 3 weeks old. The order of bleaching kinetics was as follows: PEO-20 (k=0.10 s⁻¹)>PEO-25 (k=0.083 s⁻¹)>PEO-30 (k=0.05 s⁻¹). These rate constants of SPO-doped nanostructured PEO lenses compared well to that of SPO-doped PMMA (prepared by MMA polymerization) (k=0.04 s⁻¹). SPO-doped PMMA, which exhibited the fastest response reported so far for solid-state matrices, showed a response time that was an order of magnitude slower than that achieved for the SPO-doped nanostructured PEO lenses.

In the nanostructured materials investigated here, the SPO was located within the organic domains based on the conclusions that the response was very much faster than that observed in the non-nanostructured polymeric material (i.e. PMMA) that was reported previously. Secondly, upon drying, pure PMMA would become colored and begin to exhibit reverse photochromism. Samples that were stored at ambient conditions for varying periods after their synthesis were analyzed and there was no indication of reverse photochromism. Another interesting feature was evident from the fits of the bleaching curves for the SPO-doped materials. Fresh samples (PEO-20) that has been stored for ˜40 h showed a slightly slower response (k=0.06 s⁻¹) as compared than a 3 week-old sample (k=0.10 s⁻¹). This was in contrast to the pure PMMA system, whereby the matrix environment became more restricted with aging, and the open molecular forms became stabilized by contact with the pore walls. In our case, the increased rate of photobleaching with increasing water content suggested that the nanoporous environment reduced the number of sites available in the polymer matrix domains for the open forms of the SPO to stabilize, resulting in faster bleaching kinetics. This also reflected a homogeneous dye environment at the nanometer-scale, confirming that the photochromic species were located predominantly within the organic domains of the biphasic nanocomposites.

In summary, that nanostructured bicontinuous microemulsions were shown to be excellent hosts for photochromic dyes. The SPO dye investigated showed direct photochromism, becoming colored upon UV illumination and was bleached thermally back to their colorless closed forms in the absence of UV irradiation. The response times of SPO-doped lens materials were very fast, amongst the best values reported so far for solid state composites. The materials also showed long-term stability, with no obvious competition between direct and reverse photochromism over time. As these nanostructured photochromic materials derived from bicontinuous microemulsion could be processed easily into any desired shape, they would be of interest for applications as ophthalmic lenses and optical devices

### Example 2

The following describes methods used in combination with Example 1.

The polymer membrane morphology was studied with field emission scanning electron microscopy (FESEM) (JEOL 6700). The membranes were freeze-fractured in liquid nitrogen to expose their cross-sections. Prior to examination, they were vacuum dried at room temperature for 24 h, and then coated with a thin layer of gold (JEOL ion-sputter JFC-1100). The thermal behavior of the polymer samples (˜10 mg each) was evaluated for 30-600° C. (ramp=10° C./min) under dry nitrogen flow using a Perkin Elmer TGA7 thermal gravimetric analyzer. To measure the water content of the polymer membranes, pre-weighed dry samples were immersed in deionized water at various temperatures. After the excess surface water was removed with a piece of filter paper, the weight of each fully swollen sample was recorded. The wt % of water was determined using the following equation:

EWC (%)=(Wₛ−W_{d})/W_{d}×100

where W_{d} refers to the dry sample weight before swelling, and Wₛ is the wet sample weight after immersing in water for at least 24 h. The strain, Young's modulus and tensile strength of the polymer membranes were measured by an Instron 4502 microforce tester. Samples of standard size were used according to ASTM 638. The light transmission of the polymer membranes was examined by Agilent 5453 UV-visible spectrophotometer. Refractive indices of materials, fully hydrated in phosphate buffered saline (PBS), were measured with a refractometer. Oxygen permeabilities of the materials were measured by Rehder 201T permeometer.

UV-visible absorption spectra were obtained using an Agilent 5453 UV-visible spectrophotometer. The UV source of irradiation was a 20-W Hg lamp (Philips) with a maximum wavelength of 365 nm. First, the polymer films were irradiated with the Hg lamp, the absorbance spectra were recorded until the maximum absorbance decreased to that of the non-irradiated films; the variation in maximum absorbance was plotted against time. Next, the polymer films were irradiated multiple times until no change in the photochromic properties of the polymer films was recorded. The UV light excitation was performed for 5-10 min; the plots of maximum absorbance with time were obtained. Rate constants for the thermally activated reaction to convert from the open form back to the closed form of SPO was determined by monitoring the temporal change in the absorption at 620 nm of the open form.

### Example 3

The following describes the preparation and characterization of non-limiting photochromic polymeric materials of the present invention. The two materials employed in this example have formulations as provided in Table 2.

TABLE 2
Formation of SPO-doped microemulsion-derived nanostructured polymers.

| Composition of bicontinuous | Material | Material |
|---|---|---|
| microemulsion (wt %) | A | B |
| PEO-R-MA-40 | 20.0 | 18.2 |
| GMA | 17.0 | 14.1 |
| HEMA | 43.0 | 37.9 |
| Water | 20.0 | 18.2 |
| Ethyleneglycol dimethacrylate (EGDMA) | 1.0 | 8.8 |
| 2,2-azobis[2-(2-imidazolin-2- | 0.3 | 2.7 |
| yl)propane]dihydrochloride (AIPH) | | |
| SPO | 0.1 | 0.1 |

In this example, Material A was formed using 200 mg PEO-R-MA-40, 170 mg GMA, 430 mg HEMA, 200 uL (microliters) water, 10 mg EGDMA, 3 mg AIPH, and 1 mg SPO, and Material B was formed using 200 mg PEO-R-MA-40, 156 uL GMA, 418 uL HEMA, 200 uL water, 97 uL EGDMA, 30 mg AIPH, and 1 mg SPO.

The materials were prepared as follows. PEO-R-MA-40, GMA, and HEMA were vortexed to form a first mixture. Water, EGDMA, and AIPH were then added to the first mixture, and addition vortexing was carried out until a second mixture was formed. SPO was added to the second mixture, and the resulting material was sonicated in ice to form a third mixture. Any solid material from the third mixture was separated from the third mixture (e.g., by centrifuging, for example, 30 seconds at 5 ref). The liquid portion of the third mixture was isolated and poured into molds. The liquid portion of the third mixture was left of polymerize overnight at 60° C. The unpolymerized material (e.g., the liquid portion of the third mixture) may be stored at 4° C. with no or essentially no loss of function. Material B exhibited about 10 times the tensile strength and 100 times the tensile modulus of Material A (see FIG. 13).

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those of ordinary skill in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed:

1. A method of forming a polymeric material, comprising:
polymerizing a bicontinuous microemulsion comprising water, a monomer, and a surfactant copolymerizable with said monomer, to form a porous polymeric material comprising a polymer matrix defining interconnected pores at least partially filled by water, wherein said microemulsion further comprises a photochromic agent.

2. A photochromic polymeric material for use in an ophthalmic device, comprising:
a polymer matrix defining interconnected pores, said interconnected pores containing water and said polymer matrix being substantially hydrophobic; and
wherein the polymeric material further comprises a photochromic agent.

3. An optical device, comprising a photochromic agent rendering the device switchable from a first, relatively transparent state to a second, at least partially opaque state, whereby transmission of visible light through the optical pathway can change by at least 50 percent upon switching of the device from the first state to the second state, and from the second state to the first state, each within a period of time of no more than 30 seconds upon exposure to appropriate electromagnetic radiation and/or thermal relaxation.

4. The material of claim 2, wherein the polymeric material is formed from a bicontinuous microemulsion comprising a monomer, a surfactant copolymerizable with the monomer, and water.

5. The method of claim 1, wherein said pores have a pore diameter between about 10 and about 100 nm.

6. The method of claim 1, wherein the proportion of said water is from about 15% to about 50% by weight, the proportion of said monomer is from about 5% to about 40% by weight, and the proportion of said surfactant is from about 10% to about 50% by weight.

7. The method of claim 1, wherein said microemulsion further comprises a cross-linker.

8. The method of claim 7, wherein the cross-linker is EGDMA.

9. The method of claim 1, wherein said microemulsion further comprises a polymerization initiator.

10. The method of claim 9, wherein said polymerization initiator is a photo-initiator.

11. The method of claim 10, wherein the photo-initiator is DMPA.

12. The method of claim 11, wherein said polymerizing comprises subjecting said microemulsion to ultraviolet radiation.

13. The method of claim 1, wherein said monomer is ethylenically unsaturated.

14. The method of claim 1, wherein said monomer is methyl methacrylate (MMA), 2-hydroxyethyl methacrylate (HEMA), or a combination of MMA and HEMA.

15. The method of claim 1, wherein said surfactant is a non-ionic surfactant.

16. The method of claim 1, wherein said surfactant is a poly(ethylene oxide)-macromonomer.

17. The method of claim 1, wherein the surfactant is ω-methoxy poly(ethylene oxide)₄₀ undecyl α-methacrylate macromonomer.

18. A polymeric article formed in accordance with the method of claim 1.

19. The method of claim 1, wherein said microemulsion further comprises at least one drug.

20. The method of claim 19, wherein said drug is an ophthalmic drug.

21-38. (canceled)
